(19) United States
(12) Patent Application Publication
(10) Pub. No.: US 2006/0051739 A1
(43) Pub. Date: Mar. 9, 2006

(54) Methods and composition for visualizing and interfering with chromosomal tethering of extrachromosomal molecules

(75) Inventors: Teru Kanda, Sapporo (JP); Geoffrey M Wahl, San Diego, CA (US); Michele Fasel-Otter, Corcelles (CH)

Correspondence Address: SALK INSTITUTE FOR BIOLOGICAL STUDIES;OFFICE OF TECHNOLOGY MANAGEMENT, P.O. BOX 85800, SAN DIEGO, CA, 92186-5800, US

(73) Assignee: Salk Institute for Biological Studies, LaJolla, CA (US)

(21) Appl. No.: 10/398,586

(22) PCT Filed: Sep. 7, 2001
(86) PCT No.: PCT/US01/28130

§ 371 Date: Aug. 20, 2004

Related U.S. Application Data
(60) Provisional application No. 60/230,730, filed on Sep. 7, 2000.

(51) Int. Cl. C12Q 1/70; C07H 21/04; C07H 21/02

(52) U.S. Cl. 435/5; 536/23.72

(57) ABSTRACT

The invention provides methods and compositions for visualizing and interfering with chromosomal tethering and segregation of extrachromosomal molecules.

### BACKGROUND OF THE INVENTION

The replication and subsequent faithful segregation of duplicated chromosomes are crucial for the proper transmission of the cellular genome to daughter cells. In higher eukaryotes, the nuclear membrane breaks down at the beginning of mitosis, and subsequently spindle microtubules attach to centromeric kinetochores to assure the even distribution of sister chromatids. At the end of mitosis, the nuclear membrane reassembles around each group of chromosomes to form two daughter nuclei. Therefore, it would be reasonable to assume that acentric DNA molecules should not be maintained stably in nuclei as they do not attach to microtubules, and that acentric DNA molecules should be dispersed throughout the cytoplasm subsequent to nuclear membrane breakdown.

In many cases, however, acentric DNA molecules, lacking functional centromeres, exhibit a surprisingly high stability in dividing cells. Examples of such stably-transmitted acentric DNA molecules in human cells include cellular acentric chromosomes called double minute chromosomes (DMs) and extrachromosomally replicating viral DNAs. DMs are cancer specific genomic anomalies known to harbor amplified oncogenes and drug resistance genes (Alitalo and Schwab, 1986; Hahn, 1993; Wahl, 1989). They are autonomously replicating, acentric, atelomeric, circular chromatin bodies, and usually 1-2 megabase pairs in size. Although they apparently lack functional centromeres (Barker and Hsu, 1978; Levan and Levan, 1978), their segregation efficiency is much higher than expected (Kimmel et al., 1992; Pauletti et al., 1990). Clues to the mechanisms underlying the efficient segregation came from light and electron microscopic observations showing that DMs frequently associated with mitotic chromosomes (Barker and Hsu, 1978; Hamkalo et al., 1985; Jack et al., 1987; Levan and Levan, 1978). These observations were extended using a fusion protein of human histone H2B and Aequorea victoria green fluorescent protein (H2B-GFP) to reveal DM clusters tethered to segregating daughter chromosomes in living cancer cells (Kanda et al., 1998). Time-lapse microscopy demonstrated that DMs could ‘hitchhike’ on segregating chromosomes from anaphase to telophase, indicating how chromosome tethering could contribute to increased segregation efficiency.

There is a continuing need for improved visualization of viral and cellular acentric extrachromosomal molecules that tether to chromosomes. There is a further need to identify compounds that effectively interfere with the chromosomal tethering of these viral and cellular acentric extrachromosomal molecules.

Abbreviations: Epstein-Barr virus (EBV); lac operator (lacO); Epstein-Barr nuclear antigen-1 (EBNA-1); green fluorescent protein (GFP); yellow fluorescent protein (YFP); red fluorescent protein (RFP); cyan fluorescent protein (CFP); Chinese hamster ovary cells (CHO); double minute chromosome (DM); lac repressor (lacR); fluorescent in situ hybridization (FISH); ampicillin (Amp); family of repeats (FR).

### SUMMARY OF THE INVENTION

The invention provides a preselected nucleic acid molecule comprising an extrachromosomal molecule operably linked to a tag. The extrachromosomal molecule can be any molecule that segregates with cellular chromosomes during cell division. For example, the extrachromosomal molecule may be a double minute chromosome or a viral nucleic acid sequence from a DNA or RNA virus. The viral genome may be maintained as an episome within a cell. Examples of sources of the viral nucleic acid include, but are not limited to Flaviviridae, Retroviridae, Hepadnaviridae, Papovaviridae, Adenoviridae, Herpesviridae, Poxviridae, a Hepatitis C virus, a Papillomavirus, an Epstein-Barr virus, an Influenza virus or a Polyomavirus. Alternatively, the extrachromosomal molecule may be an oncogene, such as sis, erbB, fins, sea, kit, ros, mpl, eyk, erbA, H-ras, K-ras, crk, src, abl, fps, fes, fgr, yes, mos, raf, mil, akt, jun, fos, myc, myb, ets, rel, maf, ski or qin.

The tag includes any nucleic acid sequence that encodes a selection marker (such as a blasticidin resistance gene (bsr), ampicillin, rifampicin, chloramphenicol or other art recognized drug resistance selection marker gene). Such selection markers may be driven by any operably linked promoter, such as the SRα promoter. The tag may be a reporter gene (such as green fluorescent protein, yellow fluorescent protein, red fluorescent protein or cyan fluorescent protein) that allows the extrachromosomal molecule to be detected. The tag may also be a binding site for a detectable trans-acting element that binds to the tag and thereby allows detection of the extrachromosomal molecule. The tag can be operably linked to the extrachromosomal molecule through integration of an integrating vector, into the extrachromosomal molecule.

The preselected nucleic acid may further contain an integrating vector that specifically labels double-minute chromosomes (DMs). The vector may contain Epstein-Barr virus (EBV), bovine papillomavirus (BPV), or Kaposi's sarcoma associated herpesvirus (KSHV) sequences. The vector may contain an EBNA-1 gene and an oriP sequence, wherein the oriP sequence has a plurality of EBNA-1 binding sites in two distinct regions. The vector may contain a plurality of tandem repeats of a lac operator (lacO). The vector may contain a nucleotide sequence that interferes with chromosomal tethering such as an antisense message to a tethering protein, such as the Epstein-barr nuclear antigen. The vector may encode a fusion protein that binds to an extrachromosomal molecule such as a lac repressor (lacR)-GFP fusion protein that binds with high affinity to the lacO that may be integrated into the extrachromosomal molecule.

Examples of the present invention include, but are not limited to, a plasmid vector containing a retroviral vector, a gene encoding GFP fused to lac repressor-nuclear localization signal, or a vector containing a gene encoding YFP fused to lac repressor-nuclear localization signal, or a histone H2B gene fused to a CFP gene.

The present invention further provides a chromosomal tethering polypeptide. The polypeptide can operably link a cellular chromatid and an extracellular molecule. Such an extracellular molecule may be an oriP-containing vector. This polypeptide may be of cellular or viral origin. The tethering protein may also be a peptidomimetic or a fusion polypeptide having a chromatid binding domain and a domain that binds to an extrachromosomal molecule.

The present invention further provides a method of visualizing chromosomal tethering of extrachromosomal molecules, such as viral acentric extrachromosomal molecules (DAE) or double-minute chromosomes (DM), by contacting a vector of the invention with a cell suspected of containing a DM or a DAE. The extrachromosomal molecule, such as the DM or DAE, may associate with a cellular chromatid through the action of a tethering polypeptide.

The present invention provides a method of interfering with chromosomal tethering of extrachromosomal molecules, such as viral or cellular acentric extrachromosomal molecules, by administering a vector that specifically labels the extrachromosomal molecule, such as a double-minute chromosome (DM). Additionally, the vector may produce a product that inhibits the expression or function of a tethering polypeptide, such as an antisense message to the Epstein-Barr nuclear antigen or the herpesvirus latent nuclear antigen.

The present invention also provides a method to identify an agent that modulates segregation of extrachromosomal molecules into daughter cells following division of a parent cell. Such methods can be used to identify agents that are useful for treating cancer, viral infections or other afflictions that involve extrachromosomal molecules.

The present invention also provides a method of treating cancer or viral infections by administering a pharmaceutical composition containing a compound that inhibits the tethering of viral or cellular acentric extrachromosomal molecules to a chromosome.

The present invention further comprises a chromosomally integrating vector that specifically labels DMs. The vector may be an Epstein-Barr virus (EBV), bovine papillomavirus (BPV), or Kaposi's sarcoma associated herpesvirus (KSHV) vector sequences. The vector may contain an EBNA-1 gene and an oriP sequence, wherein the oriP sequence has a plurality of EBNA-1 binding sites in two distinct regions. Further, the vector may contain a plurality of tandem repeats of a lac operator (lacO). The vector may contain a reporter gene, such as GFP or YFP, and/or may contain a selection marker, such as a blasticidin resistance gene (bsr) driven by SR promoter. The vector may further contain a nucleotide sequence that interferes with chromosomal tethering. The vector may contain a lac repressor (lacR)-GFP fusion protein that binds with high affinity to the lacO. The vector may be a modified virus, such as a modified animal virus. It may be a DNA virus, such as a member of the Herpesviridae, Papovaviridae or Adenoviridae.

The present invention provides a plasmid vector comprising retroviral vector, a gene encoding GFP fused to lac repressor-nuclear localization signal. It also provides a plasmid vector comprising retroviral vector, a gene encoding YFP fused to lac repressor-nuclear localization signal. Moreover, the present invention provides a plasmid vector comprising a histone H2B gene and a CFP gene.

### BRIEF DESCRIPTION OF THE DRAWINGS

FIG. 1. EBV vector used for specific labeling of DMs. The EBV-lacO vector contains the EBNA-1 gene and oriP sequence, which has total of 24 EBNA-1 binding sites in two distinct regions (family of repeats and dyad symmetry) (Reisman et al., 1985). The vector has 256 tandem repeats of the lac operator (lacO), to which lac repressor (lacR)-GFP fusion protein binds with high affinity. The vector also has a blasticidin resistance gene (bsr) driven by SRα promoter as a drug selection marker.

FIG. 2. DMs are preferred targets for EBV vector integration. FIG. 2A: FISH analyses demonstrating the co-localization of lacO repeats with DMs. DMs are observed as small scattered dots in mechanically spread chromosomes of colcemid-treated cells. In two color photography, signals detected using a c-myc cosmid probe were visualized as green, and lacO repeat probe were visualized as red. Chromosomes were counter-stained with DAPI and were visualized as blue. Intrachromosomal c-myc loci, lacking any overlapping red signals, are shown by arrowheads in the merged image. The scale bar is 10 μm.

FIG. 2B: Frequency of co-localization of DMs and lacO repeats. Paired black dots represent DMs with overlapping lacO signals. Fifty metaphase spreads were examined for co-localization, and they were divided into three categories; complete overlap (left), partial overlap (middle), and no overlap (right). The numbers of metaphase spreads in each category are shown.

FIG. 2C: FISH analyses using chromatin fibers prepared from untransfected COLO320DM cells (top) and cells transfected with the EBV-lacO vector (bottom). In a colored photograph, the lacO signals were visualized as red, and c-myc signals were visualized as green on the same chromatin fibers. DAPI was visualized as blue in the colored photograph. The scale bar is 10 μm.

FIG. 2D: Genomic DNAs (10 μg) of parental COLO320DM cells (lanes 1 through 3, ten copies and 1 copy equivalent of EBV-lacO plasmids added in lanes 1 and 2, respectively) and lacO integrated cells (lane 4) were digested with HindIII, which clipped out an 11 kb fragment containing the lacO repeats from the EBV-lacO plasmid. Digested DNA samples were analyzed by Southern blotting using ³²P-labelled lacO repeat (SalI-XhoI fragment of pSV2-dhfr 8.32) (Robinett et al., 1996) as a probe. An arrow indicates the expected size of the fragment containing the lacO repeats.

FIG. 3. Localization of DM-integrated and extrachromosomal EBV vectors. FIGS. 3A, 3C: Prometaphase chromosome rosettes of COLO320DM cells, having either EBV-lacO vectors integrated into DMs after stable transfection (FIG. 3A) or transiently-transfected EBV-lacO vectors (FIG. 3C), are shown. Green fluorescent dots representing the lacR-GFP protein recruited to the lacO repeats in the vectors were visualized in the colored photograph. Chromosomes were counterstained with DAPI (blue) in the colored photographs. FIGS. 3B, 3D: In a color photograph, the localization of EBNA-1 protein in the same cells as FIGS. 3A and 3C was visualized by indirect immunofluorescence as red. FIG. 3E: Distribution of native (unlabelled) DMs in an anaphase cell of the parental COLO320DM cell line. Note that DMs make clusters and attach to mitotic chromosomes when cells are not treated with colcemid. FIG. 3F: Distribution of lacO/lacR-GFP labelled DMs in an anaphase cell of the established cell line. The scale bar is 10 μm.

FIG. 4. Mitotic behavior of DMs, centromeres, and microtubules In a colored photograph, cells containing labelled DMs visualized as green were processed for immunofluorescence analyses while preserving the fluorescence of lacR-GFP. Centromeres (visualized as red in A, C, E, G, and I) and microtubules (visualized as red in B, D F, H, and J) in the same cells were detected by indirect immunofluorescence. In a color photograph, chromosomes were counterstained with DAPI (blue). DMs associating with distal chromosomal arms, but not with telomeres, are shown by arrows (C, D). DMs being incorporated into micronuclei are shown by arrows (I, J). The scale bar is 10 μm.

FIG. 5. Real time observation of DM behavior using different fluorescent proteins. (A) CFP and YFP can be used for dual color labeling. Entire chromosomes, including DMs, were stained with H2B-CFP (left), while lacO-tagged DMs are specifically stained with lacR-YFP (middle). Merged image is shown in right. The scale bar is 10 μm. (B) Time lapse images of DM behavior at metaphase-anaphase transition during mitosis. Dual color images were collected at the indicated time points (min). The scale bar is 10 μm.

FIG. 6. Microtubule inhibitors disrupt the peripheral localization of Dms Cells with lacO/lacR-GFP labelled DMs, treated with either taxol (A) or vinblastine (B), were processed for immunofluorescence analyses to detect microtubules (red) in a colored photograph while preserving the fluorescence of lacR-GFP (green) in a color photograph. Chromosomes were counterstained with DAPI (blue) in a color photograph. Note that DMs are no longer at the periphery but still associating with chromosomes. The scale bar is 10 μm.

FIG. 7. Model to explain the difference of the dynamics of various acentric DNA molecules. For simplicity, only one pair of mono-oriented prometaphase sister chromatids is drawn in each panel. The four panels show: (A) the behavior of normal mono-oriented sister chromatids; (B) a chromosome arm severed by laser microsurgery (Rieder et al., 1986); (C) a pair of DMs attached to the tip of chromosomes (large white circles); and (D) EBV vectors randomly associating with chromosomes (small white circles). A spindle pole is shown as a gray circle, and microtubules are shown as solid lines. Microtubule-mediated forces are shown as black arrows, while interacting forces between DMs and chromosomes are shown as white arrows (C). The size of each arrow represents the relative strength of each force.

### DETAILED DESCRIPTION OF THE INVENTION

Recent studies revealed that chromosome tethering may be a common mechanism for enhancing the transmission of extrachromosomally replicating viruses into daughter nuclei (Bastien and McBride, 2000; Ilves et al., 1999; Lehman and Botchan, 1998; Marechal et al., 1999; Skiadopoulos and McBride, 1998). One of the best characterized episomal vectors is based on the Epstein-Barr virus (EBV) replicon, which utilizes the cis-acting oriP sequence and the virally-encoded EBNA-1 protein (Mackey and Sugden, 1999). OriP is composed of two clusters of EBNA-1 binding sites referred to as the family of repeats and the dyad symmetry element (Reisman et al., 1985). It has been shown that EBNA-1 both enables autonomous replication of oriP-containing plasmids in human cells (Yates et al., 1985) and mediates the nuclear retention of the plasmids (Krysan et al., 1989; Middleton and Sugden, 1994). Since EBNA-1 protein localizes on mitotic chromosomes (Grogan et al., 1983; Marechal et al., 1999; Petti et al., 1990), it is reasonable to infer that EBNA-1 could recruit oriP plasmids to mitotic chromosomes. Consistent with this, fluorescence in situ hybridization (FISH) previously demonstrated that EBV vectors did associate with mitotic chromosomes (Simpson et al., 1996; Westphal et al., 1998). Such chromosome tethering should facilitate the efficient segregation of EBV vectors into daughter nuclei when the nuclear membrane reforms at the end of mitosis.

The observation of viral association with host chromosomes, “hitchhiking,” raised the intriguing question of whether DMs achieve efficient segregation by a similar mechanism. This possibility was explored by introducing EBV vectors into DM-harboring cells. Unexpectedly, EBV vectors frequently integrated into DMs subsequent to DNA transfection and selection. This observation enabled a method to preferentially integrate exogenous DNA into DMs. Cell lines with DM-EBV chimeras were derived in which lac operator (lacO) repeats were introduced as part of the EBV vector. These lacO-tagged DMs were readily detected using a fusion protein between the lac repressor (lacR) and green fluorescent protein (GFP), as previously demonstrated for visualizing homogeneously stained regions in CHO cells (Robinett et al., 1996). This provided a powerful tool for analyzing the mitotic behavior of DMs. Different distributions of free EBV vectors and DM-EBV chimeras were found, although they both hitchhiked onto mitotic chromosomes. Possible molecular mechanisms governing the behavior of these acentric molecules are discussed and described herein.

### DEFINITIONS

A “cis-acting” element or sequence refer to DNA or RNA sequences whose function require them to be on the same molecule. An example of a cis-acting element is an origin of replication.

An “extrachromosomal molecule” of the invention includes nucleic acid molecules which segregate during cell division through interaction with cellular chromatin. Examples of such extrachromosomal molecules include, but are not limited to, viral acentric extrachromosomal molecules, viral genomes and double-minute chromosomes. Extrachromosomal molecules also include nucleic acid constructs containing a nucleic acid sequence that allows interaction of the nucleic acid construct with cellular chromatin and provides for segregation of the nucleic acid construct with the cellular chromatin during cell division. Such nucleic acid sequences include origins of replication, such as oriP and the like, as well as fragments of such origins such as the FR element of oriP. It is well within the skill in the art to identify such sequences and link such sequences into nucleic acid constructs of the invention. Such constructs can also include retroviral vectors, plasmids, phagemids, yeast artificial chromosomes, bacterial artificial chromosomes and the like.

The term “modulate” or “modulates” means an increase or decrease in the occurrence of an event. For example, an agent that modulates the segregation of an extrachromosomal molecule in a cell will either increase or decrease the efficiency of extrachromosomal molecule segregation to sister cells following division of a cell treated with the agent.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

A “polypeptide” of the invention includes proteins found in nature as well as poly-amino acids that are not found in nature. Such polypeptides include fusion proteins as well as poly-amino acids that contain non-natural amino acids. Persons of skill in the art realize that many amino acid derivatives exist that can be linked into a chain of amino acids in a desired manner and that fusion proteins can be designed through recombinant techniques. The terms “polypeptide,” “protein” and “peptide” are used synonymously herein.

A “reporter gene” is a nucleic acid that expresses a detectable polypeptide. Examples of such reporter genes include those that encode for green fluorescent protein, yellow fluorescent protein, red fluorescent protein, cyan fluorescent protein, or other polypeptides that may be detected.

A “selection marker” is a nucleic acid sequence that confers resistance to a chemical, such as a drug, to a cell. Examples of such chemicals include chloramphenicol, ampicillin, rifampicin, blasticidin or the like.

A “tag” of the invention includes a marker that is associated with an extrachromosomal molecule and that allows detection of an extrachromosomal molecule. Examples of a tag include, but are not limited to, a reporter gene that expresses a polypeptide that can be detected, such as green fluorescent protein, yellow fluorescent protein, red fluorescent protein, cyan fluorescent protein, or other detectable polypeptides. A tag can also be a nucleic acid sequence that encodes a selection marker such as chloramphenicol, ampicillin, rifampicin, blasticidin resistance or the like. Such nucleic acid sequences are well known in the art. A tag can also be a cis-acting element that is recognized by a trans-acting element. An example of a cis-acting element is a nucleic acid sequence that encodes a repressor binging site, such as the lac-repressor binding site found within the lac operator (lacO). This cis-acting element can be integrated into an extrachromosomal molecule and bound by a detectable trans-acting element that allows detection of an extrachromosomal molecule. An example of such a trans-acting element is a fusion polypeptide of a repressor polypeptide, such as the lac repressor (lacR), and a detectable polypeptide, such as green fluorescent protein. Such trans-acting factors may also include a nuclear localization signal that will cause the fusion polypeptide to localize to the nucleus. It will be recognized that there are many combinations of such cis-acting and trans-acting elements that are within the scope of the invention.

A “tethering polypeptide” is a polypeptide that provides an association of an extrachromosomal molecule with a cellular chromatid. A tethering polypeptide may comprise a cellular protein or a viral protein. Examples of such a tethering polypeptide are the Epstein-Barr Nuclear Antigen-1 protein (EBNA-1) and the Herpesvirus late nuclear antigen (LANA).

A “recombinant tethering polypeptide” is a tethering polypeptide produced through recombinant methods. An example of such a recombinant polypeptide is a protein having the N-terminal cellular chromatid binding domain of EBNA-1 linked to the C-terminal oriP binding domain of EBNA-1 through an recombinantly produced amino acid linkage. Alternatively, such a polypeptide can have the N-terminal cellular chromatid binding domain of EBNA-1 fused to a domain that would bind to a recombinantly designed cis-acting element within an extrachromosomal molecule. An example of such a recombinant polypeptide is a protein having the N-terminal cellular chromatid binding domain of EBNA-1 linked to the lac repressor binding domain of the lac repressor protein.

A “trans-acting” element refers to a polypeptide or nucleic acid whose function requires them not to be directly linked to the same molecule. An example includes a polypeptide that binds to a cis-acting element that is operably linked to an extrachromosomal molecule.

I. A Preselected Nucleic Acid Molecule Comprising an Extrachromosomal Molecule Operably Linked to a Tag.

The invention provides a preselected nucleic acid molecule comprising an extrachromosomal molecule operably linked to a tag. It is contemplated that the extrachromosomal molecule is any molecule that is tethered to a cellular chromatid during cellular division. Such tethering allows the extrachromosomal molecule to be segregated into progeny cells upon division of the parent cell. The invention provides a method to tag the extrachromosomal molecule which allows the extrachromsomal molecule to be detected during chromosome segregation and cell division. The tag may become operably linked to the extrachromosomal molecule through integration of an integrating vector, which contains the tag, into the extrachromosomal molecule. Thus, the invention provides integrating vectors that are able to integrate into an extrachromosomal molecule and operably link a tag into the extrachromosomal molecule.

Extrachromosomal molecules: The extrachromosomal molecule can be any molecule that segregates with cellular chromosomes during cell division. Examples of such extrachromosomal molecules include, but are not limited to, double minute chromosomes, viral acentric extrachromosomal molecules, cellular acentric extrachromosomal molecules and recombinantly engineered extrachromosomal molecules.

Double minute chromosomes, viral acentric extrachromosomal molecules and cellular acentric extrachromosomal molecules are well known in the art. Recombinantly engineered extrachromosomal molecules include, but are not limited to, plasmid and viral based vectors into which a nucleic acid sequence has been inserted that allows the extrachromosomal molecule to associate with a cellular chromatid and be segregated into daughter cells during cell division. Nucleic acid sequences that may be operably linked into extrachromosomal molecules include, but are not limited to, an origin of replication such as EBV-oriP, or nucleic acid sequences that are able to interact with tethering proteins, such as the FR element of EBV-oriP. A recombinantly engineered extrachromosomal molecule can also contain nucleic acid sequences that allow the molecule to be detected within a cell. Such nucleic acid sequences include reporter genes that encode fluorescent proteins or selection markers that confer chemical, i.e. drug, resistance to a cell. It is contemplated that a recombinantly engineered extrachromosomal molecule may be introduced into a cell such that it is segregated into progeny cells during division of the parent cell. It is further contemplated that such extrachromosomal molecules may be constructed to associate with recombinantly engineered tethering polypeptides, described below. This allows for the production of recombinantly engineered extrachromosomal molecules and tethering polypeptides that specifically associate with each other and allow specific interactions of the recombinant extrachromosomal molecules with chromatids to be assessed.

Tag: A tag includes any nucleic acid sequence which provides for detection of an operably linked extrachromosomal molecule. A tag may confer a detectable trait onto an extrachromosomal molecule. Examples of traits that may be conferred include selection markers and reporter genes. Selection markers include genes that confer resistance to a chemical or drug such as blasticidin, ampicillin, rifampicin or chloramphenicol. Reporter genes encode a detectable polypeptide such as a fluorescent protein as described herein. Additionally, a tag may function as a cis-acting binding site for a detectable trans-acting element that binds to the tag when the tag has integrated into an extrachromosomal molecule. Binding of the detectable trans-acting element to the cis-acting tag, that is operably linked to the extrachromosomal molecule, allows the extrachromosomal molecule to be detected. Binding sites for trans-acting elements include nucleic acid sequences to which a detectable trans-acting element will bind and thereby tag the extrachromosomal molecule containing the binding site. There are many combinations of binding sites and trans-acting elements that are well known in the art. These combinations are exemplified by the lac repressor binding site (lacO) and the lac repressor (lacR). Thus, a cis-acting lac repressor binding site may be operably linked to an extrachromosomal molecule through integration of an integrating vector, which contains the tag, into the extrachromosomal molecule. A trans-acting fusion protein having the lac repressor fused to a fluorescent protein can bind to the cis-acting lac repressor binding site and allow the extrachromosomal molecule to be detected. Such methods are within the scope of the invention and are well known to those of skill in the art.

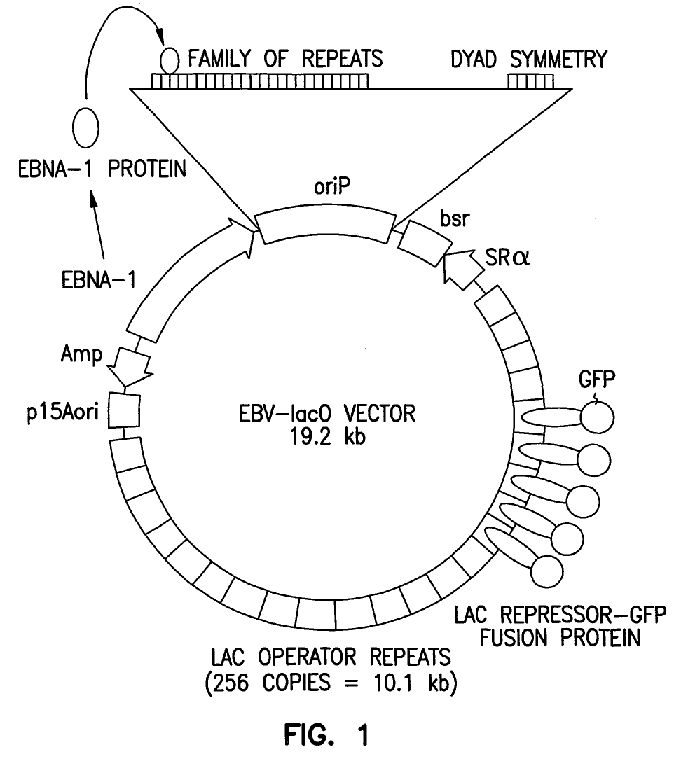

Integrating vector: The invention provides vectors that can integrate into the extrachromosomal molecules discussed above. The vectors of the invention may be derived from a plasmid, virus, retrovirus, phagemid or other cloning vehicles and vectors known in the art. Such vectors may include a nucleic acid sequence that promotes the insertion of the vector into an extrachromosomal molecule. In one embodiment, the vectors of the invention include Epstein-Barr nucleic acid sequences, like that encoding the origin of replication (oriP). Optionally, such vectors may encode the ENBA-1 protein, lac operator repeats and lac repressor-fluorescent protein fusion protein individually or in combination. One example of a vector of the invention is the EBV-lacO vector (FIG. 1).

Chromosomal tethering polypeptide: The invention provides chromosomal tethering polypeptides. A tethering polypeptide of the invention includes those that promote the association of an extrachromosomal molecule with a cellular chromatid such that the extracellular molecule is segregated with the cellular chromatid during cell division.

Tethering polypeptides can be isolated according to the methods of the invention or be created through recombinant DNA technology according to methods known in the art. Such recombinant molecules include, but are not limited to, a fusion polypeptide having a chromosome binding domain linked to an extrachromosomal molecule binding domain. An example of a tethering polypeptide is a polypeptide having the N-terminal chromosome binding domain of EBNA-1 linked to the C-terminal oriP binding domain of EBNA-1. In another example, a tethering polypeptide can have the N-terminal chromosome binding domain of EBNA-1 linked to any domain that is able to bind to a site within an extrachromosomal molecule. This type of tethering polypeptide is exemplified by a fusion protein having the N-terminal chromosome binding domain of EBNA-1 fused to a lac repressor.

Cells: The invention provides cells that contain an extrachromosomal molecule that has an operably linked tag as described above. The cells of the invention include, but are not limited to, mammalian cells, insect cells, bacteria, yeast and any other cell which can contain an extrachromosomal molecule that segregates with a cellular chromatid during cell division. Examples of cells which may be used include 3T3, BHK21, MDCK, HeLa, PtK1, L6, PC12, COLO320DM and SP2 cells. Additional cells may be obtained from the American Type Culture Collection. (Hay et al., eds., American Type Culture Collection Catalogue of Cell Lines and Hybridomas, 6^{th} ed. Rockville, Md.: American Type Culture Collection, 1988). These cells may be grown under any condition that allows them to divide. Conditions for cell and tissue culture are well known in the art. Ham, Proc. Natl. Acad. Sci. USA 53:288 (1965); Loo et al., Science, 236:200 (1987); Sato et al., eds. Growth of Cells in Hormonally Defined Media. Cold Spring Harbor, N.Y.: Cold Spring Harbor Laboratory (1982).

II. A Method to Visualize Chromosomal Tethering of an Extrachromosomal Molecule to a Cellular Chromatid.

The invention provides a method to visualize tethering of an extrachromosomal molecule to a cellular chromatid. The method involves tagging the extrachromosomal molecule and visually detecting the tag in order to determine whether the extrachromosomal molecule is tethered to a cellular chromatid. The extrachromosomal molecule may be tagged by contacting the molecule with a vector of the invention such that the vector integrates into the molecule. Integration of the vector into the extrachromsomal molecule incorporates a cis-acting element into the extrachromosomal molecule. Such cis-acting elements may include, but are not limited to, nucleic acid sequences to which trans-acting elements bind and thereby tag the extrachromosomal molecule. In one embodiment, the extrachromosomal molecules are tagged with a fluorescent polypeptide. The fluorescent polypeptide may then be visualized through use of a microscope as described herein.

The present method may be used to screen for agents that modulate chromosomal tethering and segregation of extrachromosomal molecules as described herein. Alternatively, the above method may be used as a diagnosis tool to determine if cells contain extrachromosomal molecules that are segregated during cell division. During such a diagnosis scheme, cells could be obtained through biopsy of tissue suspected of containing extrachromosomal molecules. These cells would then be contacted with a vector of the invention that would integrate into and tag extrachromosomal molecules contained within the suspect cells. The cells could then be cultured and the presence of extrachromosomal molecules could then be determined. Such a method would be useful because extrachromosomal molecules are known to encode oncogenes and drug resistance genes that cause proliferative disorders.

III. A Method to Interfere with Tethering of Extrachromosomal Molecules to a Cellular Chromatid.

The present invention also provides a method for treating a test cell that contains a tagged extrachromosomal molecule with a candidate agent and then determining if the candidate agent inhibits tethering of the extrachromosomal molecule to a cellular chromomatid. In one embodiment, the method involves use of a cell that contains a fluorescently tagged extrachromosomal molecule. Such molecules can be produced according to the methods described herein. A control cell is used that has the tagged extrachromosomal molecule but that is not treated with a candidate agent. A test cell has a tagged extrachromosomal molecule and is treated with a candidate agent to produce a treated cell. The control cell and the treated cell are allowed to begin cell division and the linkage of the extrachromosomal molecule in the test cell and the control cell is then determined and compared. If a candidate agent interferes with tethering, the extrachromosomal molecule will not be associated with a cellular chromatid through cell division. In one embodiment, the extrachromosomal molecule may be fluorescently tagged according to the methods described herein and the effects of a candidate agent on tethering can be determined through use of fluorescent microscopy as described herein.

It is understood that any trait that can be integrated into an extrachromosomal molecule and detected in order to determine the tethering of an extrachromosomal molecule to a cellular chromatid is within the scope of the invention.

IV. A Method to Identify an Agent that Modulates Segregation of Extrachromosomal Molecules into Daughter Cells during Division of a Parent Cell.

The method involves treating a test cell that contains a tagged extrachromosomal molecule with a candidate agent and then determining if the candidate agent increases or decreases segregation of the extrachromosomal molecule during cell division when compared to a control cell. In one embodiment, the method involves use of a cell having a fluorescently tagged extrachromosomal molecule. Methods to prepare a fluorescently tagged extrachromosomal molecule are disclosed herein. Segregation of the fluorescently tagged extrachromosomal molecule can then be followed through use of fluorescence microscopy. Alternatively, fluorescent activated cell sorting (FACS) can be used to determine segregation efficiency. Through use of FACS, cells containing an extrachromosomal molecule can be sorted from those that do not. This method provides an automated procedure to rapidly screen numerous candidate agents for their ability to inhibit or increase segregation of extrachromosomal molecules.

Alternatively, an extrachromosomal molecule having a selection marker may be used within the method of the invention. In this embodiment, a cell having an extrachromosomal molecule that encodes a selective marker is treated with a test agent to produce a test cell. The test cell is allowed to undergo cell division and is then subjected to selection according to the selection marker. A untreated control cell having an extrachromosomal molecule that encodes the selective marker is allowed to divide in parallel and then is subjected to selection according to the selection marker. The number of resistant cells of the test cell and the control cell is then compared to determine if the candidate agent increased or decreased segregation of the extrachromosomal molecule.

It is understood that any trait that can be integrated into an extrachromosomal molecule and detected in order to determine segregation efficiency of the extrachromosomal molecule is within the scope of the invention.

V. A method of Treating Cancer or Viral Infection.

The invention provides a method of treating cancer or viral infection. The method involves contacting cells with an agent that interferes with the tethering of extrachromosomal molecules to a cellular chromatid which causes loss of the extrachromosomal molecule upon cell division. Such agents can be identified according to the methods disclosed herein and administered to an animal in need thereof according to methods well known in the pharmaceutical arts.

The agents of the invention may be formulated into a variety of acceptable compositions. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts are obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids also are made.

The compounds may be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### EXAMPLE I

### Materials and Methods

Plasmids: EBV oriP and EBNA-1 coding sequences derived from pCEP4 (Invitrogen) and a blasticidin resistance gene (Izumi et al., 1991) derived from pYN3215-bsr (kindly provided by Dr. Fumio Hanaoka, Osaka University) were subcloned into pMBL19 to make pMBL19-EBVbsr. pMBL19, which has a bacterial p15A ori, was chosen for its ability to subclone unstable inserts (Nakano et al., 1995). Lac operator (lacO) repeats (256 direct repeats) derived from pSV2-dhfr 8.32 (Robinett et al., 1996) were subcloned into the pMBL19-EBVbsr to make EBV-lacO vector using STBL2 competent cells (Life Technologies, Grand Island, N.Y.) (Belmont et al., 1999).

pCLMFG-lacR-GFP was constructed by subcloning a gene encoding EGFP (Clontech, Palo Alto, Calif.) fused to lac repressor-nuclear localization signal (p3′SSdimerClonEGFP) (Robinett et al., 1996) into a splicing retroviral vector pCLMFG-MCS (kindly provided by Dr. Nikunj Somia, Salk Institute), a derivative of the pMFG vector (Dranoff et al., 1993). pCLMFG-lacR-YFP was constructed in the same way using EYFP gene (Clontech).

A histone H2B-CFP fusion gene was made by swapping the GFP gene of H2B-GFPN1 (Kanda et al., 1998) with the ECFP gene (Clontech). The H2B-CFP gene was subcloned into a pCLNRX vector (Naviaux et al., 1996).

Production of VSV-G pseudotyped retroviruses was performed by co-transfection of each retroviral vector and pMD.G (the plasmid encoding the envelope protein VSV-G) into 293 gp/bsr cells as described (Miyoshi et al., 1997).

Establishing cell lines with lacO-tagged DMs: COLO320DM cells harboring DMs containing an amplified c-myc gene were grown as described (Kanda et al., 1998). Exponentially growing cells (1×10⁷) were transfected with 5 μg of the EBV-lacO vector using electroporation (BioRad, Hercules, Calif.), resuspended in 10 ml of culture medium, and plated into two 10 cm dishes (8 ml, 2 ml for each dish). Blasticidin (15 μg/ml, Calbiochem, San Diego, Calif.) was added to the transfected cells 24 hours after transfection, and cells were selected for 14 days. Drug resistant cells were further grown under reduced blasticidin concentration (5 μg/ml). Blasticidin resistant colonies were isolated 4 weeks after transfection and then re-plated into 48 well dishes. Cells were expanded in media containing blasticidin (5 μg/ml) and 12 fast growing clones were selected for infection with the lacR-GFP retrovirus. Punctate staining in nuclei was observed in all clones analyzed, and three independent clones which exhibited the brightest fluorescent dots by lacR-GFP staining were chosen for further FISH analyses as these clones were expected to contain the highest number of EBV-lacO vectors. This strategy was repeated independently three times to confirm the reproducibility of the experimental data.

Fluorescence in situ hybridization (FISH): Cells were treated with colcemid (100 μg/ml) for 50 minutes, and chromosome spreads were prepared by conventional fixation. For dual color FISH, c-myc cosmid DNA was labelled with biotin, while the lacO repeat (SalI-XhoI fragment of pSV2-dhfr 8.32) (Robinett et al., 1996) was labelled with digoxigenin using random prime labeling. Denaturation, hybridization, and washing were performed as previously described (Shimizu et al., 1996). Signals were detected using FITC-avidin (10 μg/ml, Vector Laboratories, Burlingame, Calif.) and rhodamine-conjugated sheep anti-digoxigenin antibody (4 μg/ml, Boehringer Mannheim, Indianapolis, Ind.). Chromosomes were counterstained with 4′,6′-diamidino-2-phenylindole (DAPI, 1 μg/ml) in VectaShield (Vector).

Chromatin fibers were prepared on slide glasses as previously described for fiber-FISH (Parra and Windle, 1993). Signals were detected using 3 sequential steps of a signal amplification protocol as follows: (1) FITC-avidin (5 μg/ml) and anti-digoxigenin monoclonal antibody (1 μg/ml, Boehringer Mannheim); (2) biotinylated goat anti-avidin (1 μg/ml, Vector) and digoxigenin-labelled sheep anti-mouse IgG (2 μg/ml, Boehringer Mannheim); (3) FITC-avidin (5 μg/ml) and rhodamine-conjugated sheep anti-digoxigenin antibody (4 μg/ml).

Visualizing DM-integrated and free EBV vectors by in vivo lacR-GFP staining: One of the established cell lines, containing DMs tagged with lacO repeat, was infected with lacR-GFP retrovirus. The infected cells were subcloned by limited dilution to obtain sublines in which DMs are more uniformly labelled with lacR-GFP. For time lapse imaging, the same cell line was infected with H2B-CFP and lacR-YFP viruses simultaneously, and the double-labelled cells were subcloned by limited dilution.

For visualizing EBV vectors in transiently transfected cells, a subline of COLO320DM cells stably expressing lacR-GFP protein was established by retrovirus infection and subcloning of lacR-GFP positive cells. The established cells were transfected with the EBV-lacO vector by electroporation, and the transfected cells were harvested for immunofluorescence analyses at 3 days posttransfec

Immunofluorescence staining: Cells were harvested by gentle pipetting, attached to slide glasses by cytospin (500 rpm, 1 minute, Shandon, Pittsburgh, Pa.), and fixed with 3.7% formaldehyde for 10 minutes. When indicated, cells were treated with either taxol (10 μM paclitaxel, Sigma Chemical Co., St. Louis, Mo.) or vinblastine (10 μg/ml, Sigma) for 3 hours prior to harvesting them in order to disrupt microtubules. Slides were washed with PBS three times, and treated with blocking buffer (2.5% BSA, 0.2M glycine, 0.1% TritonX-100) for 30 minutes.

Primary and secondary antibodies were diluted in the blocking buffer. Primary antibodies were rabbit anti EBNA-1 serum K67-3 (1:1000, kindly provided by Dr. Jaap Middeldorp, Free University Hospital, Amsterdam, the Netherlands), human CREST autoantiserum hACA-M (for detecting centromeres, 1:2000 dilution) (Sullivan et al., 1994), and monoclonal anti α-tubulin (for detecting microtubules, 1:2000 dilution, Sigma). Following incubation for 60 minutes at room temperature, glass slides were washed three times with PBS. Secondary antibodies were Texas-Red conjugated anti-rabbit IgG (1:500, Jackson ImmunoResearch, West Grove, Pa.), Cy5 conjugated goat anti-human IgG (1:1000, Amersham, Piscataway, N.J.), and rhodamine conjugated anti mouse IgG (1:1000, Boehringer Mannheim), respectively. Following incubation for 60 minutes at room temperature, slides were washed three times with PBS, and chromosomes were counterstained with DAPI (1 μg/ml). Fluorescence of lacR-GFP was preserved well by this protocol.

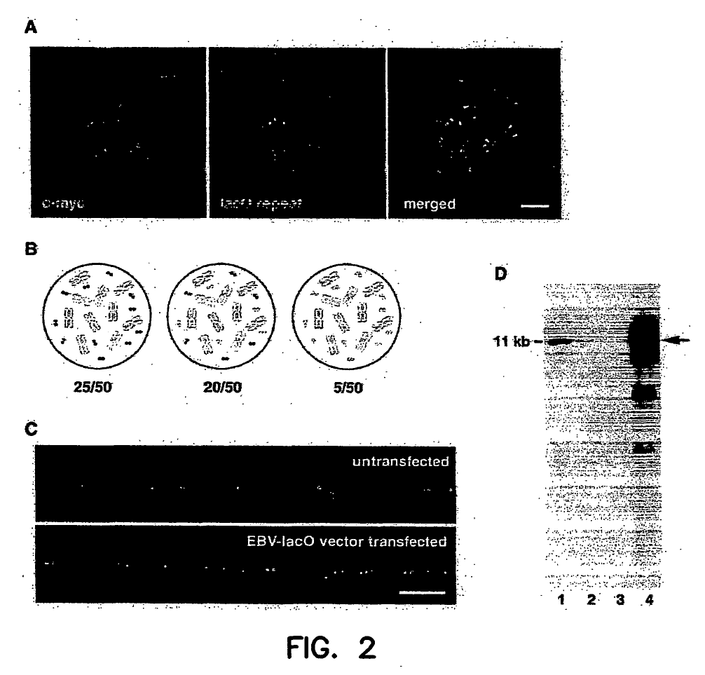

Microscopy: All images appearing in the Figures were collected using a DeltaVision microscope system (Applied Precision Inc. Issaquah, Wash.) with either a 63×/NA 1.4 or a 100×/NA 1.35 oil immersion objective. For fixed specimens (except for the images of FIG. 2), three-dimensional data sets were collected to visualize EBV vectors and DMs as they distributed in multiple focal planes. Optical sections were collected at 0.2-μm focal intervals; pixel size was 0.111 μm for 63× objective and 0.0669 μm for 100× objective. Out-of-focus contamination was removed from each optical section via deconvolution processing and two-dimensional images were created by projecting the three-dimensional data stacks using the software supplied with the DeltaVision system.

For observation of living specimens, cells were grown on 40 mm cover slips pretreated with fibronectin (25 μg/ml in PBS) and placed in an FCS2 chamber system (Bioptechs, Butler, Pa.) with prewarmed medium (containing 20 mM HEPES pH 7.3). Special filter sets required for CFP and YFP detection (Ellenberg et al., 1999) were installed into the DeltaVision microscope system. Single slice images were collected every 2 minutes using 100× objective equipped with an objective heater (Bioptechs). Pixel size was 0.1338 μm and a binning factor of 2 was used to minimize the total exposure time during observation. Pseudo-color images were created using Adobe Photoshop (Adobe Systems, San Jose, Calif.).

### Results

### DMs are Preferred Targets for EBV Vector Integration

FISH analyses revealed that stably transfected EBV vectors frequently co-localized with DMs. One explanation of this finding was that EBV vectors integrated into DMs. To facilitate the tracking of transfected EBV vectors, two hundred and fifty-six direct repeats of the lac operator (lacO) were added to the EBV vector (EBV-lacO) (FIG. 1). If the EBV-lacO vectors recombined with DMs, the DMs were tagged with lacO repeats and were rapidly detected in cells expressing a fusion protein between the lac repressor and GFP (lacR-GFP) (FIG. 1) (Robinett et al., 1996). The EBV-lacO vector was transfected into COLO320DM cells, which contain DMs encompassing c-myc loci, and then blasticidin resistance encoded by the EBV vector was selected for. Drug resistant colonies were obtained at frequencies of approximately 1×10⁻⁴˜10⁻⁵. It should be noted that both the oriP sequence and the EBNA-1 gene were required for obtaining transformants, as no colonies arose in transfections employing vectors lacking either element (transformation efficiency <1×10⁻⁷). Three drug resistant colonies were expanded into cell lines and analyzed by dual color FISH analyses using a c-myc cosmid probe (to detect DMs) and a lacO repeat DNA (to visualize EBV-lacO vectors). The result revealed that, in all three clones, the signals generated by both probes frequently overlapped in metaphase chromosome spreads (FIG. 2A). Analysis of 50 metaphase spreads of one of the established clones showed that 50% (25/50) of the spreads exhibited complete colocalization of lacO/DM signals, and 90% (45/50) of the metaphase spreads contained at least one pair of DMs with overlapping lacO signals (FIG. 2B). On the average, each chromosome spread contained 18.3 (±19.2) pairs of DMs, and 12.7 (±13.2) DMs had overlapped lacO signals. It is noteworthy that lacO signals never overlapped with intrachromosomal c-myc signals (FIG. 2A), and no evidence of EBV-lacO integration into chromosomes was observed in >50 metaphase spreads in three different clones. Thus, the combined data strongly suggest that the majority of transfected EBV vectors preferentially co-localize with DMs.

Direct evidence was obtained that EBV-lacO vectors integrated into DMs using FISH analysis of stretched chromatin fibers (Parra and Windle, 1993). This analysis revealed that arrays of lacO signals were detected on the same DNA fibers exhibiting c-myc signals (FIG. 2C). Semi-quantitative Southern blotting revealed approximately 130 copies of lacO repeats per cell, and several bands in addition to those of the expected-size were detected (FIG. 2D). As there are approximately 13 DMs with integrated lacO repeats per cell (see above), it was concluded that each DM contains approximately 10 copies of EBV-lacO (i.e., 130/13). The complex patterns observed by fiber-FISH, together with the extra bands observed in Southern blotting, indicate that the integration events are likely to be complex, or that further rearrangements occurred subsequent to the initial integration.

### EBV-lacO/DM Chimeras and Free EBV Vectors Behave Differently in Prometaphase Cells

The established cell lines containing the lacO-integrated DMs were infected with retrovirus expressing the lacR-GFP fusion protein. Retroviral infection resulted in readily detectable lacR-GFP protein expression in approximately 80% of the recipient cells, and approximately 20% of which exhibited punctate fluorescent dots representing DMs. The lacR-GFP expressing populations were subcloned to yield clones in which 60% of the cells had punctate fluorescent DMs.

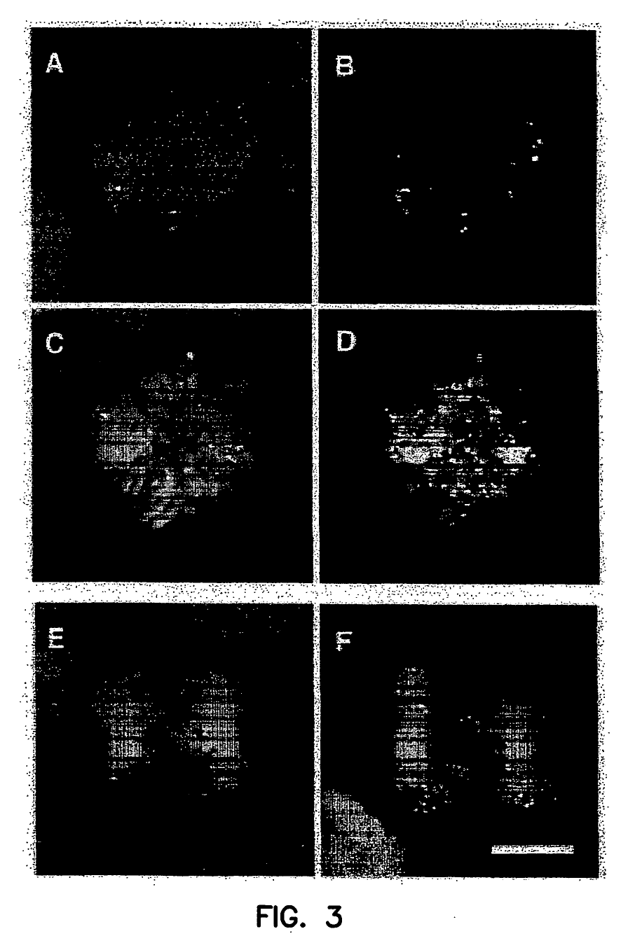

The distribution of EBV-lacO/DM chimeras in prometaphase cells, in which chromosomes aggregate briefly into a single, wheel-shaped ring called a chromosome rosette (Nagele et al., 1995) was first focused on. It was found that the fluorescent dots always attached to the periphery of the chromosome rosette (FIG. 3A). Immunofluorescence analyses revealed that EBNA-1 protein colocalized with EBV-integrated DMs (FIG. 3B), indicating that EBNA-1 was recruited to the oriP sequences that had been integrated into DMs. The mitotic distribution of EBV-lacO vectors after transient transfection into COLO320DM cells (see methods) was also examined. It was inferred that at 3 days posttransfection, most EBV plasmids were not integrated, and one or a few DMs may have contained integrated EBV plasmids. The fluorescent dots representing the transiently transfected EBV-lacO vectors were found to associate randomly with prometaphase chromosomes (FIG. 3C), corresponding well with previous FISH results indicating no preferential peripheral localization (Simpson et al., 1996; Westphal et al., 1998). In this case, EBNA-1 staining was rather diffuse on chromosomes, as observed previously (Grogan et al., 1983; Petti et al., 1990), but still colocalized with the EBV vectors (FIG. 3D). These data are consistent with free EBV-lacO vectors associating randomly with mitotic chromosomes, while DM-integrated EBV vectors localizing at the periphery. Importantly, it was found that native DMs and EBV-integrated DMs displayed equivalent mitotic behavior (FIG. 3E, F), although the latter had co-localizing EBNA-1 protein (FIG. 3B). Therefore, it is likely that the mitotic behavior of the chimeric extrachromosomal molecules faithfully represents that of native DMs. These observations justify the use of EBV-lacO/DMs as a tool to analyze DM dynamics.

### Mitotic Behaviors of DMs, Centromeres, and Microtubules

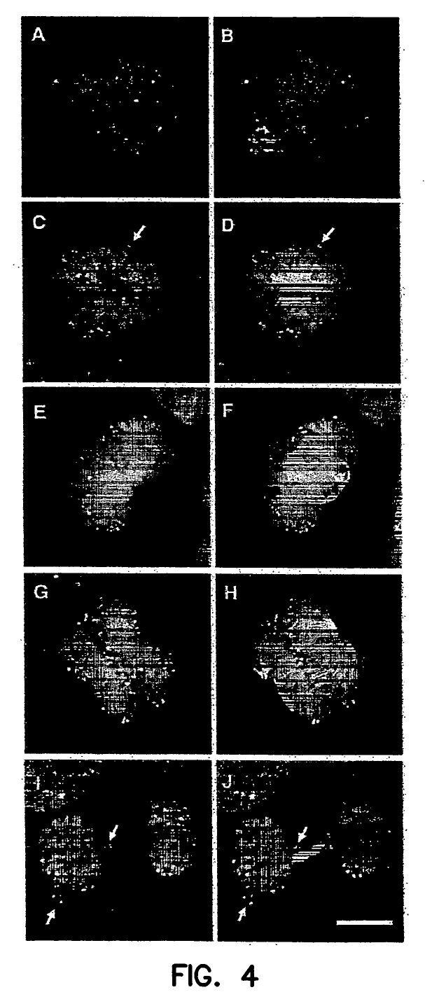

The fluorescent labeling strategy for DMs described above enables visualization of DMs, centromeres, and microtubules simultaneously in various phases of mitosis. Centromeres and spindle microtubules were detected by indirect immunofluorescence while preserving the fluorescence of lacR-GFP. This analysis confirmed that DMs lack centromeric antigens (Barker and Hsu, 1978; Levan and Levan, 1978) (FIG. 4A, C, E, G, I), and that DMs do not associate with kinetochore microtubules (FIG. 4D, F, H). DMs and centromeres exhibited distinctly different behaviors during the phases of mitosis. In interphase cells (FIG. 4A, B), DMs and centromeres were dispersed independently in nuclei. However, in prometaphase cells (FIG. 4C, D), paired fluorescent DM dots attached to the periphery of the chromosome rosettes (Nagele et al., 1995), as described above, while centromeres localized centrally as they were pulled inward by the attached microtubules. In metaphase cells (FIG. 4E,F), DMs did not strictly align on metaphase plates, but rather associated with the periphery of the aligned chromosomes. At the metaphase-anaphase transition (FIG. 4G,H), centromeres of sister chromatids were pulled apart by the attached spindle microtubules. In contrast, some DMs were still clearly observed as double dots in anaphase cells, demonstrating that the connections between sister minute chromosomes are not readily broken at the metaphase-anaphase transition. The majority of DMs, still attached to the distal chromosomal arms, lagged behind centromeric regions that were pulled toward opposing poles in anaphase cells (FIG. 4G,H). In telophase cells (FIG. 4I, J), most DMs were incorporated into daughter nuclei along with the chromosomes, while a minority became entrapped in micronuclei (Shimizu et al., 1996; Tanaka and Shimizu, 2000).

Real Time Observation of DM Behavior using Dual-Color Fluorescent Protein Labeling

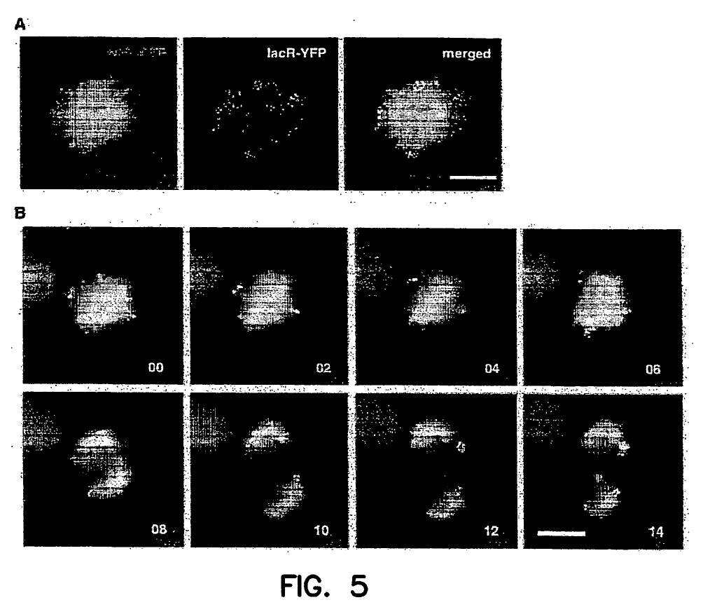

The above results with fixed cells could mask movements occurring in living cells, and it does not provide a dynamic view of DM behavior. These concerns were addressed by taking advantage of the in vivo expression of distinguishable fluorescent fusion proteins (Ellenberg et al., 1999) in order to analyze the dynamics of DMs and chromosomes in living mitotic cells. Cells with lacO-labelled DMs were simultaneously infected with two different retroviruses expressing either H2B-CFP or lacR-YFP. H2B-CFP should label chromosomes and DMs, as described previously (Kanda et al., 1998), while lacR-YFP should label only DMs as descried above. Chromosomes and DMs in the same cell were observed with minimal spectral overlap (FIG. 5A).

Representative time lapse images demonstrating the behavior of DMs at the metaphase-anaphase transition are shown (FIG. 5B). DMs were found at the tips of chromosome arms in late prometaphase cells (time 00). Subsequently, paired sister chromatids were observed to align on metaphase plates (time 04). When sister chromatids started to separate at the onset of anaphase (time 06), DMs quickly changed their position and lagged behind segregating chromosomes (time 08, 10). In early G1 phase, significant numbers of DMs were still observed as paired dots (data can be seen in the attached movie), confirming the previous observation obtained by a premature chromosome condensation protocol (Takayama and Uwaike, 1988). These time lapse images correspond very well with the data of fixed cells (FIG. 4). Taken together, the peripheral localization of DMs in prometaphase chromosome rosettes and their lagging behavior in anaphase cells strongly suggest that DMs may be repelled from the spindle poles.

### Microtubule Inhibitors Disrupt DM Peripheral Localization

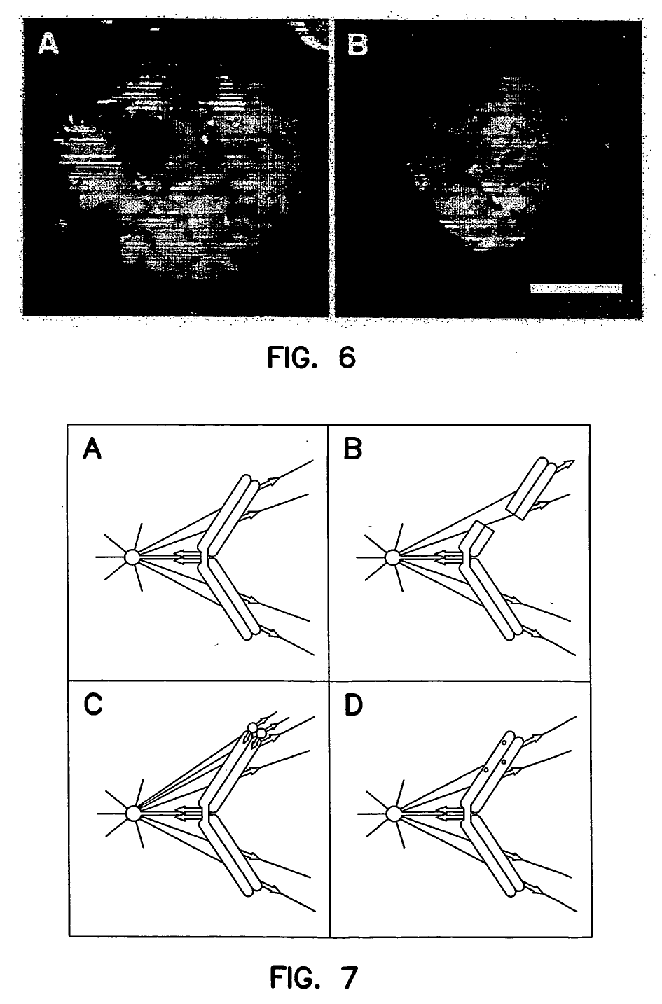

The ability to readily visualize DMs without using FISH protocol has enabled the investigation of the mechanisms that contribute to their behavior during mitosis. The possibility that DMs are held away from the spindle poles by the microtubule-mediated antipolar force, to which normal chromosome arms are also known to be subjected (Fuller, 1995; Rieder et al., 1986) was examined. DM-labelled cells were treated with either a microtubule stabilizer (taxol) or destabilizer (vinblastine). DMs and microtubules were visualized by lacR-GFP and immunofluorescence staining, respectively. Microtubules of taxol-treated cells showed multiple aster-like structures (FIG. 6A), while vinblastine-treated cells exhibited rod-like microtubules (FIG. 6B). In both cases, chromosome organization was completely disrupted and DMs were no longer attached to the periphery of clustered chromosomes. Rather, DMs distributed randomly, although they were still associated closely with chromosomes (FIG. 6). These results support the idea that DMs are repelled from the spindle poles via microtubule-mediated antipolar forces and that microtubules do not mediate the attachment of DMs to mitotic chromosomes.

### Discussion

The mechanisms underlying the precision of chromosome segregation are being elucidated with increasing detail. It now appears that some autonomously replicating DNA viruses achieve high efficiency segregation not merely by their high copy number, but rather by having devised strategies to associate with chromosomes (Bastien and McBride, 2000; Ilves et al., 1999; Lehman and Botchan, 1998; Marechal et al., 1999; Skiadopoulos and McBride, 1998). The data herein highlights the role of chromosomes as “cargo ships” on which both viral replicons and cellular DMs are loaded to enable their efficient transmission to daughter nuclei.

It was determined that EBV vectors integrate into DMs at high frequency. This targeted integration of EBV vectors into DMs was totally unexpected since EBV vectors containing oriP and the EBNA-1 gene are usually maintained as extrachromosomal elements without integrating into chromosomes (Yates et al., 1985). It was found that EBV vectors randomly associated with mitotic chromosomes as well as DMs after transient transfection (FIG. 3C). This observation corresponds well with the known noncovalent association of EBV vectors with mitotic chromosomes (Harris et al., 1985; Marechal et al., 1999; Simpson et al., 1996; Westphal et al., 1998). However, after stable transfection into DM-harboring cells, it was observed that EBV vectors recombined with DMs, and that the chimeric molecules of EBV vectors and DMs were always found at the periphery of mitotic chromosomes (FIG. 3A). Free extrachromosomal EBV vectors randomly associating with mitotic chromosomes after stable transfection were not detected. Interestingly, it was found that the same EBV-lacO vector could be maintained extrachromosomally in stably transfected HeLa cells, which do not contain DMs (data not shown). Therefore, the EBV vectors appear to remain as independent extrachromosomal molecules in cells without DMs, while there is a high probability of integration of multiple EBV plasmids into DMs.

The molecular basis of preferential integration into DMs remains a mystery. It is possible that DMs and normal chromosomes have different tendencies to undergo recombination. Another possibility is that EBV replicons and DMs share the same replication machinery during S phase of the cell cycle, which increases the probability of recombination between replication intermediates. This would be consistent with the observed requirements for both oriP and EBNA-1 to recombine with DMs. The heterogeneity in the number and fraction of DMs containing integrated EBV-lacO sequences (FIG. 2B) is most easily explained if it is assumed that a single integration event occurred at an early stage of selection shortly after transfection. Replication of the EBV-lacO/DM chimeras, followed by their unequal selection, could have led to eventual emergence of clones with dissimilar numbers of chimeric extrachromosomal molecules. EBV vectors are thought to provide a general strategy for tagging DMs derived from different chromosomal loci as EBV-DM chimeras were obtained in the CRL2270 neuroblastoma line containing extrachromosomally amplified N-myc amplicons (data not shown).

The chimeric extrachromosomal molecules of DMs and EBV-lacO vectors appear to exhibit the same behavior as native (unlabelled) DMs. The visualization strategy involving lacR-GFP can be used in combination with immunofluorescence, as it does not require harsh denaturation of DNA, and it preserves chromosomal fine structures far better than FISH (Robinett et al., 1996). This sensitive methodology enabled the visualization of DMs together with centromeres and microtubules (FIGS. 4, 6), and to track the dynamics of DMs and chromosomes in living human cells (FIG. 5). The data confirms and further extends previous analyses using fixed cells (Levan and Levan, 1978) and previous findings obtained using H2B-GFP staining (Kanda et al., 1998).

A model is proposed in which DMs are subject to two “forces” in mitotic cells (FIG. 7). First, DMs appear to be pushed away from the poles, as they are always found at the periphery of prometaphase chromosome rosettes, and they lag behind segregating chromosomes in anaphase cells. It is well known that, while the kinetochore microtubules pull chromosomes poleward, another force appears to repel chromosomal arms (Fuller, 1995) (FIG. 7A). Laser microsurgery experiments demonstrated that severed chromosomal arms immediately moved radially outward to the periphery of the aster (Rieder et al., 1986) (FIG. 7B), indicating the existence of such an astral exclusion force. It has been proposed that plus-oriented kinesin-related microtubule motor proteins, distributed along chromosomal arms, mediate this astral exclusion force (Fuller, 1995). Since DMs originate from chromosomes, in this case the distal part of chromosome 8 (8q24; c-myc locus), it is reasonable to assume that DMs are covered with such motor proteins and subject to a microtubule-mediated antipolar force (FIG. 7C). The present observations that peripheral localization of DMs became apparent only after nuclear membrane breakdown and chromosomes attached to microtubules, and that disrupting microtubule organization prevented the peripheral localization of DMs, support the model. The antipolar force working on chromosomal fragments is likely to be proportional to their size (Fuller, 1995). This can partly explain the difference between the behavior of severed chromosomal arms and that of DMs, since bigger acentric chromosome fragments should be subject to a stronger antipolar force compared to smaller DMs (FIG. 7B, C). Random chromosomal association of free EBV vectors can be explained by the lack of plus-oriented motor proteins and astral exclusion force on free EBV vectors (FIG. 7D). It is unlikely that DMs specifically associate with telomeres, as some DMs associated with chromosomal arms at positions clearly distinct from telomeric regions (FIG. 4C, D).

The second “force” acting on DMs keeps them attached to mitotic chromosomes (FIG. 7C). This force is not affected by microtubule disruption. Although there is a study demonstrating that DMs associate with chromosomes via nucleolar material (Levan and Levan, 1978), no further experimental data supporting the idea has been presented. The present finding that DMs are frequent recombinational targets of EBV vectors leads to another proposed model in which DMs may somehow mimic the behavior of viral vectors. It has been suggested that chromosome tethering of EBV vectors is mediated by the cis-acting oriP sequence and trans-acting viral protein EBNA-1 (Krysan et al., 1989; Mackey and Sugden, 1999; Marechal et al., 1999; Middleton and Sugden, 1994; Simpson et al., 1996). It was recently found that EBNA-1 appears to serve as a bridge between chromosomes and oriP-containing vectors (Kanda T et al, manuscript in preparation). This observation raises the possibility that DMs may also have cis-acting sequences that recruit cellular transacting factors to them to mediate chromosome association. This possibility is strengthened by the finding that DMs consist of multiple copies of amplicons, each copy of which contains cellular replication origin(s) that are usually associated with scaffold/matrix attachment regions (S/MAR) (Carroll et al., 1991; Pemov et al., 1998). A recent study showed that an episomal vector containing a human S/MAR sequence and SV40 origin is associated with mitotic chromosomes (Baiker et al., 2000). Therefore, it is conceivable that DMs containing multiple S/MARs attach to chromosomal scaffolds, which then gives the appearance that they are associating with mitotic chromosomes, even though there is no direct connection between chromosomes and DMs. The interacting force mediated by S/MAR-bound proteins may be strong enough to compete with the weak antipolar forces working on DMs (FIG. 7C).

The ability of viral replicons and DMs to interact with chromosomes provides a simple solution to the problem of high efficiency segregation of acentric DNA molecules. Interfering with the molecular interactions between viral replicons/DMs and mitotic chromosomes would increase the mitotic loss rate of latently infected viruses, or DMs that are providing survival or selective advantage to cancer cells. Therefore, understanding the molecular interactions that mediate such associations suggests new molecular targets for anti-viral and anti-cancer therapy.

### REFERENCES

Alitalo, K. and Schwab, M. (1986). Oncogene amplification in tumor cells. Adv. Cancer Res. 47, 235-281.
Baiker, A., Maercker, C., Piechaczek, C., Schmidt, S. B., Bode, J., Benham, C. and Lipps, H. J. (2000). Mitotic stability of an episomal vector containing a human scaffold/matrix-attached region is provided by association with nuclear matrix. Nat. Cell Biol. 2, 182-184.
Barker, P. E. and Hsu, T. C. (1978). Are double minutes chromosomes? Exp. Cell Res. 113, 456-458.
Bastien, N. and McBride, A. A. (2000). Interaction of the Papillomavirus E2 Protein with Mitotic Chromosomes. Virology 270, 124-134.
Belmont, A. S., Li, G., Sudlow, G. and Robinett, C. (1999). Visualization of large-scale chromatin structure and dynamics using the lac operator/lac repressor reporter system. Methods Cell Biol. 58, 203-222.
Carroll, S. M., Trotter, J. and Wahl, G. M. (1991). Replication timing control can be maintained in extrachromosomally amplified genes. Mol. Cell. Biol. 11, 4779-4785.
Dranoff, G., Jaffee, E., Lazenby, A., Golumbek, P., Levitsky, H., Brose, K, Jackson, V., Hamada, H., Pardoll, D. and Mulligan, R. C. (1993). Vaccination with irradiated tumor cells engineered to secrete murine granulocyte-macrophage colony-stimulating factor stimulates potent, specific, and long-lasting anti-tumor immunity. Proc. Natl. Acad. Sci. USA 90, 3539-3543.
Ellenberg, J., Lippincott-Schwartz, J. and Presley, J. F. (1999). Dual-colour imaging with GFP variants. Trends Cell Biol. 9, 52-56.
Fuller, M. T. (1995). Riding the polar winds: chromosomes motor down east. Cell 81, 5-8.
Grogan, E. A., Summers, W. P., Dowling, S., Shedd, D., Gradoville, L. and Miller, G. (1983). Two Epstein-Barr viral nuclear neoantigens distinguished by gene transfer, serology, and chromosome binding. Proc. Natl. Acad. Sci. USA 80, 7650-7653.
Hahn, P. J. (1993). Molecular biology of double-minute chromosomes. BioEssays 15, 477-484.
Hamkalo, B. A., Farnham, P. J., Johnston, R. and Schimke, R. T. (1985). Ultrastructural features of minute chromosomes in a methotrexate-resistant mouse 3T3 cell line. Proc. Natl. Acad. Sci. USA 82, 1026-1030.
Harris, A., Young, B. D. and Griffin, B. E. (1985). Random association of Epstein-Barr virus genomes with host cell metaphase chromosomes in Burkitt's lymphoma-derived cell lines. J. Virol. 56, 328-332.
Ilves, I., Kivi, S. and Ustav, M. (1999). Long-term episomal maintenance of bovine papillomavirus type 1 plasmids is determined by attachment to host chromosomes, which is mediated by the viral E2 protein and its binding sites. J. Virol. 73, 4404-4412.
Izumi, M., Miyazawa, H., Kamakura, T., Yamaguchi, I., Endo, T. and Hanaoka, F. (1991); Blasticidin S-resistance gene (bsr): a novel selectable marker for mammalian cells. Exp. Cell Res. 197, 229-233.
Jack, E. M., Waters, J. J. and Harrison, C. J. (1987). A scanning electron microscopy study of double minutes from a human tumour cell line. Cytogenet. Cell Genet. 44, 49-52.
Kanda, T., Sullivan, K F. and Wahl, G. M. (1998). Histone-GFP fusion protein enables sensitive analysis of chromosome dynamics in living mammalian cells. Curr. Biol. 8, 377-385.
Kimmel, M., Axelrod, D. E. and Wahl, G. M. (1992). A branching process model of gene amplification following chromosome breakage. Mutat. Res. 276, 225-239.
Krysan, P. J., Haase, S. B. and Calos, M. P. (1989). Isolation of human sequences that replicate autonomously in human cells. Mol. Cell. Biol. 9, 1026-1033.
Lehman, C. W. and Botchan, M. R. (1998). Segregation of viral plasmids depends on tethering to chromosomes and is regulated by phosphorylation. Proc. Natl. Acad. Sci. USA 95, 4338-4343.
Levan, A. and Levan, G. (1978). Have double minutes functioning centromeres? Hereditas 88, 81-92.
Mackey, D. and Sugden, B. (1999). Applications of oriP plasmids and their mode of replication. Methods Enzymol. 306, 308-328.
Marechal, V., Dehee, A., Chikhi-Brachet, R., Piolot, T., Coppey-Moisan, M. and Nicolas, J. C. (1999). Mapping EBNA-1 domains involved in binding to metaphase chromosomes. J. Virol. 73, 4385-4392.
Middleton, T. and Sugden, B. (1994). Retention of plasmid DNA in mammalian cells is enhanced by binding of the Epstein-Barr virus replication protein EBNA1. J. Virol. 68, 4067-4071.
Miyoshi, H., Takahashi, M., Gage, F. H. and Verma, I. M. (1997). Stable and efficient gene transfer into the retina using an HIV-based lentiviral vector. Proc. Natl. Acad. Sci. USA 94, 10319-10323.
Nagele, R., Freeman, T., McMorrow, L. and Lee, H. Y. (1995). Precise spatial positioning of chromosomes during prometaphase: evidence for chromosomal order. Science 270, 1831-1835.
Nakano, Y., Yoshida, Y., Yamashita, Y. and Koga, T. (1995). Construction of a series of pACYC-derived plasmid vectors. Gene 162, 157-158.
Naviaux, R. K., Costanzi, E., Haas, M. and Verma, I. M. (1996). The pCL vector system: rapid production of helper-free, high-titer, recombinant retroviruses. J. Virol. 70, 5701-5705.
Parra, I. and Windle, B. (1993). High resolution visual mapping of stretched DNA by fluorescent hybridization. Nat. Genet. 5, 17-21.
Pauletti, G., Lai, E. and Attardi, G. (1990). Early appearance and long-term persistence of the submicroscopic extrachromosomal elements (amplisomes) containing the amplified DHFR genes in human cell lines. Proc. Natl. Acad. Sci. USA 87, 2955-2959.
Pemov, A., Bavykin, S. and Hamlin, J. L. (1998). Attachment to the nuclear matrix mediates specific alterations in chromatin structure. Proc. Natl. Acad. Sci. USA 95, 14757-14762.
Petti, L., Sample, C. and Kieff, E. (1990). Subnuclear localization and phosphorylation of Epstein-Barr virus latent infection nuclear proteins. Virology 176, 563-574.
Reisman, D., Yates, J. and Sugden, B. (1985). A putative origin of replication of plasmids derived from Epstein-Barr virus is composed of two cis-acting components. Mol. Cell. Biol. 5, 1822-1832.
Rieder, C. L., Davison, E. A., Jensen, L. C., Cassimeris, L. and Salmon, E. D. (1986). Oscillatory movements of monooriented chromosomes and their position relative to the spindle pole result from the ejection properties of the aster and half-spindle. J. Cell Biol. 103, 581-591.
Robinett, C. C., Straight, A., Li, G., Willhelm, C., Sudlow, G., Murray, A. and Belmont, A. S. (1996). In vivo localization of DNA sequences and visualization of large-scale chromatin organization using lac operator/repressor recognition. J. Cell Biol. 135, 1685-1700.
Schwam D R, Luciano R L, Mahajan S S, Wong L, Wilson A C. Carboxy terminus of human herpesvirus 8 latency-associated nuclear antigen mediates dimerization, transcriptional repression, and targeting to nuclear bodies. Virol. September 2000; 74(18):8532-40.
Shimizu, N., Kanda, T. and Wahl, G. M. (1996). Selective capture of acentric fragments by micronuclei provides a rapid method for purifying extrachromosomally amplified DNA. Nat. Genet. 12, 65-71.
Simpson, K., McGuigan, A. and Huxley, C. (1996). Stable episomal maintenance of yeast artificial chromosomes in human cells. Mol. Cell. Biol. 16, 5117-5126.
Skiadopoulos, M. H. and McBride, A. A. (1998). Bovine papillomavirus type 1 genomes and the E2 transactivator protein are closely associated with mitotic chromatin. J. Virol. 72, 2079-2088.
Sullivan, K. F., Hechenberger, M. and Masri, K (1994). Human CENP-A contains a histone H3 related histone fold domain that is required for targeting to the centromere. J. Cell Biol. 127, 581-592.
Takayama, S. and Uwaike, Y. (1988). Analysis of the replication mode of double minutes using the PCC technique combined with BrdUrd labeling. Chromosoma 97, 198-203.
Tanaka, T. and Shimizu, N. (2000). Induced detachment of acentric chromatin from mitotic chromosomes leads to their cytoplasmic localization at G(1) and the micronucleation by lamin reorganization at S phase. J. Cell Sci. 113, 697-707.
Wahl, G. M. (1989). The importance of circular DNA in mammalian gene amplification. Cancer Res. 49, 1333-1340.
Westphal, E. M., Sierakowska, H., Livanos, E., Kole, R. and Vos, J. M. (1998). A system for shuttling 200-kb BAC/PAC clones into human cells: stable extrachromosomal persistence and long-term ectopic gene activation. Hum. Gene Ther. 9, 1863-1873.
Yates, J. L., Warren, N. and Sugden, B. (1985). Stable replication of plasmids derived from Epstein-Barr virus in various mammalian cells. Nature 313, 812-815.

The invention is described with reference to various specific and preferred embodiments and techniques. It should be understood, however, that many variations and modifications may be made while remaining within the scope of the invention. All referenced publications, patents, and patent documents are intended to be incorporated by reference, as though individually incorporated by reference.

What is claimed is:

1. A preselected nucleic acid molecule comprising an extrachromosomal molecule operably linked to a tag.

2. The preselected molecule of claim 1, wherein the extrachromosomal molecule is a double minute chromosome.

3. The preselected molecule of claim 1, wherein the extrachromosomal molecule is a viral nucleic acid sequence.

4. The vector of claim 3, wherein the viral genome is maintained as an episome within a cell.

5. The preselected molecule of claim 3, wherein the viral nucleic acid sequence is from an RNA virus.

6. The preselected molecule of claim 3, wherein the viral nucleic acid sequence is from a DNA virus.

7. The preselected molecule of claim 3, wherein the viral nucleic acid sequence is from Flaviviridae, Retroviridae, Hepadnaviridae, Papovaviridae, Adenoviridae, Herpesviridae, Poxviridae, a Hepatitis C virus, a Papillomavirus, an Epstein-Barr virus, an Influenza virus or a Polyomavirus.

8. The preselected molecule of claim 1, wherein the extrachromosomal molecule comprises an oncogene.

9. The preselected molecule of claim 8, wherein the oncogene is selected from sis, erbB, fins, sea, kit, ros, mpl, eyk, erbA, H-ras, K-ras, crk, src, abl, fps, fes, fgr, yes, mos, raf, mil, akt, jun, fos, myc, myb, ets, rel, mat, ski or qin.

10. The preselected molecule of claim 1, wherein the tag is a reporter gene.

11. The preselected molecule of claim 10, wherein the reporter gene is green fluorescent protein, cyan fluorescent protein, red fluorescent or yellow fluorescent protein.

12. The preselected molecule of claim 1, wherein the tag is a selection marker.

13. The preselected molecule of claim 12, wherein the selection marker is resistance to chloramphenicol, rifampicin, ampicillin or blasticidin.

14. The preselected molecule of claim 13, wherein the selection marker is a blasticidin resistance gene (bsr) driven by an SRα promoter.

15. The preselected molecule of claim 1, wherein the tag is a binding site for a detectable trans-acting element that binds to the tag.

16. The preselected molecule of claim 1, comprising a vector that integrates into an extrachromosomal molecule.

17. The preselected molecule of claim 16, wherein the vector comprises Epstein-Barr virus (EBV), bovine papillomavirus (BPV), or Kaposi's sarcoma associated herpesvirus (KSHV) vector sequences.

18. The preselected molecule of claim 16, comprising EBNA-1 sequences and an oriP sequence, wherein the oriP sequence has a plurality of EBNA-1 binding sites.

19. The preselected molecule of claim 18, wherein the EBNA-1 binding sites are located at two distinct regions within the vector.

20. The preselected molecule of claim 18, wherein the EBNA-1 binding sites have dyad symmetry within the vector.

21. The preselected molecule of claim 13, comprising an FR element from oriP.

22. The preselected molecule of claim 18, comprising a plurality of tandem repeats of a lac operator (lacO).

23. The preselected molecule of claim 13, further comprising a nucleotide sequence that interferes with chromosomal tethering.

24. The preselected molecule of claim 13, further comprising a polynucleotide that encodes a lac repressor (lacR)-GFP fusion protein that binds to the lac operator.

25. A preselected molecule comprising a reporter gene fused to a lac repressor-nuclear localization signal.

26. The preselected molecule of claim 25, wherein the reporter gene is green fluorescent protein, cyan fluorescent protein, red fluorescent or yellow fluorescent protein.

27. The preselected molecule of claim 26, further comprising retroviral sequences.

28. The preselected molecule of claim 26, further comprising plasmid sequences.

29. A vector comprising a histone H2B gene fused to a reporter gene.

30. The vector of claim 29, wherein the reporter gene is green fluorescent protein, cyan fluorescent protein, red fluorescent or yellow fluorescent protein.

31. The vector of claim 29, further comprising retroviral sequences.

32. The vector of claim 29, further comprising plasmid sequences.

33. A recombinant chromosomal tethering polypeptide.

34. The polypeptide of claim 33, operably linked to a cellular chromatid and an extrachromosomal molecule.

35. The polypeptide of claim 34, wherein the extrachromosomal molecule is a double minute chromosome.

36. The polypeptide of claim 33, operably linked to a cellular chromatid and to an oriP-containing vector.

37. The polypeptide of claim 33, wherein the polypeptide comprises a cellular polypeptide.

38. The polypeptide of claim 33, wherein the polypeptide comprises a viral polypeptide.

39. A cell comprising the preselected molecule of claim 1, the vector of claim 29 or the polypeptide of claim 33.

40. The cell of claim 39, wherein the cell is a COLO320DM cell.

41. A method of visualizing chromosomal tethering of an extrachromosomal molecule comprising contacting the vector of claims 13, 25, or 29, with a cell suspected of containing an extrachromosomal molecule.

42. The method of claim 41, wherein the extrachromosomal molecule is a double minute chromosome.

43. The method of claim 41, wherein the extrachromosomal molecule is a virus.

44. A method of interfering with chromosomal tethering of extrachromosomal molecule by contacting the vector of claims 13, 25, or 29, with a cell suspected of containing an extrachromosomal molecule.

45. The method of claim 44, wherein the extrachromosomal molecule is a double minute chromosome.

46. The method of claim 44, wherein the extrachromosomal molecule is a virus.

47. A method to identify at least one agent that modulates chromosomal tethering of an extrachromosomal molecule comprising:
(a) contacting a cell that contains an extrachromosomal molecule with a test agent; and
(b) determining if the agent causes an increase or decrease in segregation of the extrachromosomal molecule when the cell divides.

48. The method of claim 47, wherein the extrachromosomal molecule is operably linked to a tag.

49. The method of claim 48, wherein the tag is a reporter gene.

50. The method of claim 49, wherein the reporter gene is green fluorescent protein, cyan fluorescent protein, red fluorescent or yellow fluorescent protein.

51. The method of claim 47, wherein the extrachromosomal molecule is operably linked to a selection marker.

52. The method of claim 51, wherein the selection marker is resistance to chloramphenicol, rifampicin, ampicillin or blasticidin.

53. The method of claim 47, wherein the cell is the cell of claim 39.

54. The method of claim 47, wherein the cell comprises an extrachromosomal molecule into which a vector of claim 13 has integrated.

55. The method of claim 47, wherein the extrachromosomal molecule is a double minute chromosome or a virus.

56. A method of treating cancer comprising administering a pharmaceutical composition comprising a compound that inhibits tethering of an extrachromosomal molecule to a chromosome.

57. The method of claim 56, wherein the extrachromosomal molecule is a virus or a double minute chromosome.

58. A method of treating a viral infection comprising administering a pharmaceutical composition comprising a compound that inhibits tethering of a viral extrachromosomal molecule to a cellular chromatid.

59. A method for identifying an antiviral agent, comprising:
(a) contacting a cell comprising a viral acentric extrachromosomal molecule with a test compound; and
(b) identifying a compound that inhibits association of the viral acentric extrachromosomal molecule with a cellular chromatid.

60. The method according to claim 59, further comprising (c) determining if the compound inhibits the association of a tethering polypeptide with the cellular chromatid or with the viral acentric extrachromosomal molecule.

61. The method of claim 60, wherein the tethering polypeptide is Epstein-Barr nuclear antigen or herpesvirus latent nuclear antigen.

62. A method for identifying an anticancer agent, comprising:
(a) contacting a cell comprising an extrachromosomal molecule with a test compound; and
(b) identifying a compound that inhibits association of the extrachromosomal molecule with a cellular chromatid.

63. The method according to claim 62, further comprising (c) determining if the compound inhibits association of a tethering polypeptide with the cellular chromatid or with the extrachromosomal molecule.

64. The method according to claim 63, wherein the polypeptide is Epstein-Barr nuclear antigen or herpesvirus latent nuclear antigen.

65. A chromosomally integrating vector that specifically labels double-minute chromosomes (DMs).

66. The vector of claim 65, wherein the vector is an Epstein-Barr virus (EBV), bovine papillomavirus (BPV), or Kaposi's sarcoma associated herpesvirus (KSHV) vector sequences.

67. The vector of claim 65, wherein the vector comprises an EBNA-1 gene and an oriP sequence, wherein the oriP sequence has a plurality of EBNA-1 binding sites in two distinct regions.

68. The vector of claim 65, wherein the vector comprises a plurality of tandem repeats of a lac operator (lacO).

69. The vector of claim 65, wherein the vector comprises a reporter gene.

70. The vector of claim 69, wherein the reporter gene is GFP or YFP.

71. The vector of claim 65, wherein the vector comprises a selection marker.

72. The vector of claim 71, wherein the selection marker is a blasticidin resistance gene (bsr) driven by SR promoter.

73. The vector of claim 65, wherein the vector further comprises a nucleotide sequence that interferes with chromosomal tethering.

74. The vector of claim 65, wherein the vector further encodes a lac repressor (lacR)-GFP fusion protein that binds with high affinity to the lacO.

75. The vector of claim 65, wherein the vector is a modified virus.

76. The vector of claim 65, wherein the vector is a modified animal virus.

77. The vector of claim 65, wherein the vector is a DNA virus.

78. The vector of claim 65, wherein the vector is a member of the Herpesviridae, Papovaviridae or Adenoviridae.

79. A plasmid vector comprising retroviral vector, a gene encoding GFP fused to lac repressor-nuclear localization signal.

80. A plasmid vector comprising retroviral vector, a gene encoding YFP fused to lac repressor-nuclear localization signal.

81. A plasmid vector comprising a histone H2B gene and a CFP gene.
